(19) United States
(12) Patent Application Publication
(10) Pub. No.: US 2018/0215395 A1
(43) Pub. Date: Aug. 2, 2018

(54) CONTEXT DERIVED DRIVER ASSISTANCE

(71) Applicant: Intel Corporation, Santa Clara, CA (US)

(72) Inventors: Bernard N. Keany, Lake Oswego, OR (US); Norman Bright, Portland, OR (US)

(21) Appl. No.: 15/423,037

(22) Filed: Feb. 2, 2017

(51) Int. Cl. B60W 50/14; B60W 40/08; G06K 9/00; A61B 3/113; A61B 5/11; A61B 5/18; A61B 5/00

(52) U.S. Cl. CPC B60W 50/14; A61B 2562/0257; G06K 9/00288; G06K 9/00832; G06K 9/00604; A61B 3/113; A61B 5/1112; A61B 5/18; A61B 5/6893; B60W 2040/0809; B60W 2300/34; B60W 2420/42; B60W 2540/04; B60W 2550/406; B60W 2550/20; B60W 2550/12; B60W 2520/105; A61B 2560/0242; A61B 2562/0219; A61B 2562/0223; B60W 40/08

(57) ABSTRACT

System and techniques for context derived driver assistance are described herein. An identity of a vehicle operator of a vehicle may be determined. An operating context of the vehicle may be determined. A notification concerning the operating context may be adaptively adjusted based on the identity of the vehicle operator.

### TECHNICAL FIELD

Embodiments described herein generally relate to providing driver assistance and, specifically in some embodiments, to providing context derived driver assistance.

### BACKGROUND

There are millions of drivers on the roads today. Drivers may become distracted or may not be aware of events happening in proximity to the vehicle. Distractions and unawareness of events happening around the vehicle may result in collisions. Existing approaches for driver assistance may provide a human driver with limited types of alerts that attempt to provide cues which the driver may use to avoid a collision. The cues may be the same for all drivers regardless of the driver's experience in the current context in which the vehicle is operating.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a block diagram of an example of an environment and a system for context derived driver assistance, according to an embodiment.

FIG. 2 is a block diagram of an example of a system for context derived driver assistance, according to an embodiment.

FIG. 3 is a block diagram of an example of a machine learning system for context derived driver assistance, according to an embodiment.

FIG. 4 illustrates an example of a method for context derived driver assistance, according to an embodiment.

FIG. 5 is a block diagram illustrating an example of a machine upon which one or more embodiments may be implemented.

### DETAILED DESCRIPTION

Automobile manufacturers are including an increasing number of features into vehicles to assist drivers and improve vehicle safety. These features may include lane departure warnings, blind spot detection, pedestrian detection, crash avoidance systems, and parking assistance. The driver may be alerted through a number of visual, audio, and haptic notifications. These alerts may add to driver distraction especially when combined with an increase in vehicle infotainment features in cars. Traditional driver alert systems may function the same for all drivers of a vehicle irrespective of driving conditions. In addition to factory installed equipment delivered in new vehicles, drivers may be able to purchase smart glasses providing heads-up display (HUD) of directions and provide augmented reality experiences such as point of interest (PoI) location based alerts. The confluence of automotive systems and user added systems may increase driver distraction even more.

The presently disclosed subject matter delivers a context-driven custom experience for the driver based on a variety of factors such as attention, experience, surrounding driving conditions, and driver preference. Information from a HUD device may be used to determine a number of contexts (e.g., how and where the vehicle is operating, current state of operator such as attentiveness, etc.) such as the direction of the driver's attention through gaze tracking sensors combined with accelerometer, gyroscope, and magnetometer and/or the volume of surrounding traffic through the use of a world-facing camera that is part of the HUD. The contexts may be fused with in-vehicle sensors and cameras to create an adaptive experience customized for the user. These contexts may result in a single stream of information delivered through the most appropriate way for the experience of the driver and the surrounding driving conditions. In an example, the HUD may include world facing 3D depth cameras and user centric sensors such as accelerometer, gyroscope, magnetometer, infrared, or camera sensors to track gaze. The information from the HUD may be fused with the sensors and actuators in the vehicle to deliver an adaptive experience based on the abilities and experience of the driver combined with the characteristics of the surrounding traffic and context about the driver's familiarity with the location of the vehicle.

Rather than providing a one-size fits all approach, the systems and techniques disclosed herein provide individualized driver assistance notification based on, for example, the driver's experience, the driver's attention level, driving conditions, and the driver's familiarity with the current location of the vehicle. Notifications may be provided via multiple modalities (e.g., seat vibrations, side mirror display, etc.) that may be enabled and disabled individually or in various combinations to provide the driver with an appropriate level of driver assistance notification.

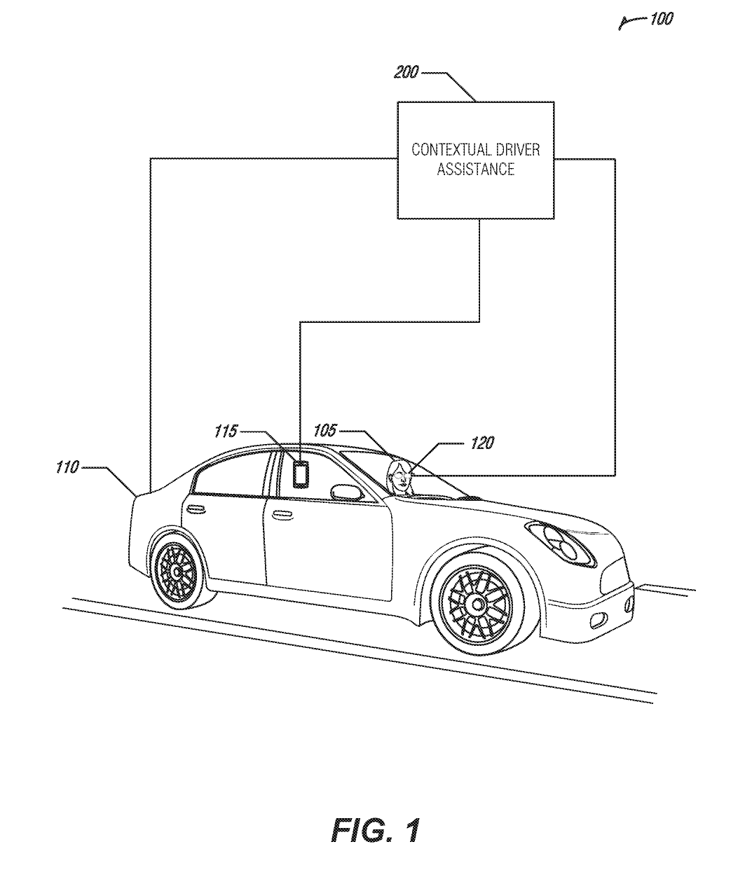

FIG. 1 is a block diagram of an example of an environment 100 and a system 200 for context derived driver assistance, according to an embodiment. The environment 100 may include a vehicle operator 105 (e.g., driver, controller, etc.) operating a vehicle 110 (e.g., car, truck, van, etc.). The vehicle operator 105 may have (e.g., operate, provide, etc.) a mobile device 115 (e.g., smartphone, tablet, in car entertainment system, etc.) and may be wearing a heads-up display (HUD) 120 (e.g., smart glasses, VR headset, etc.). The vehicle 110, mobile device 115, and the HUD 120 may be communicatively coupled (e.g., via wireless network. Bluetooth, near filed communication, etc.) to the system 200. In some examples, the system 200 may be implemented in the mobile device 115 and the vehicle 110 and the HUD 120 may be communicatively coupled to the mobile device 115. The mobile device may be inside the vehicle 110 and may be coupled (e.g., using shortwave radio, near field communication, etc.) to the vehicle 110.

The vehicle 110 may include a variety of sensors such as, for example, proximity sensors, accelerometers, gyroscopes, global positioning sensors, temperature sensors, rain sensors, etc. The sensors in the vehicle 110 may collect a variety of information about the vehicle 110 and the environment in which the vehicle 110 is operating. For example, the vehicle 110 sensors may detect traffic levels, traffic volumes, traffic density, weather conditions, etc. The sensor data may be used by a variety of vehicle operator assistance systems that may provide collision avoidance alerts, lane departure alerts, etc. The sensor data from the sensors in the vehicle 110 may be accessible through an interface to an on-board computer of the vehicle 110 (e.g., via on-board diagnostics (OBD), OBD II, controller area network (CAN) bus, near field communication, shortwave radio, etc.).

The HUD 120 may be worn by the vehicle operator 105 and may include a variety of sensors such as, for example, three-dimensional cameras, infrared cameras, accelerometers, gyroscopes, magnetometers, proximity sensors, etc. The HUD 120 may include a display that is visible by the vehicle operator 105 without obscuring the field of view of the vehicle operator 105. The sensors in the HUD 120 may be used to detect context information such as, for example, the direction of the gaze of the vehicle operator 105, traffic volume, etc.

The mobile device 115 may include a variety of sensors such as, for example, global positioning system sensor, accelerometer, gyroscope, camera, microphone, magnetometer, etc. The mobile device 115 may include a display device (e.g., screen, touch screen, etc.). The sensors of the mobile device 115 may be able to gather context data describing conditions inside the vehicle 110 which may include information about the vehicle operator 105. The mobile device 115 may provide access to internet delivered information such as, for example, traffic conditions for the route of the vehicle 110. The internet delivered information may be used to derive contextually aware routing decisions.

Sensor data from the vehicle 110, mobile device 115, and HUD 120, may be transmitted to the system 200 to determine a context in which the vehicle 110 is being operated. For example, the context may include weather conditions, speed at which the vehicle 110 and/or traffic surrounding the vehicle 110 is traveling, attentiveness of the vehicle operator 105, infotainment features (e.g., radio, navigation system, etc.) of the vehicle 110 in use, location of the vehicle, etc. The context may describe a scenario such as, for example, that the vehicle 110 is in line at a drive-up window, stuck in traffic, etc. The system 200 may employ a variety of machine learning and/or other data analytic techniques to identify the context. For example, previous sensor data may be processed (e.g., using supervised and/or unsupervised training techniques, etc.) to extract features corresponding to a variety of contexts. The sensor data from the vehicle 110, the mobile device 115, and the HUD 120 may be processed to extract features which may be matched to models of the various contexts to identify the context in which the vehicle 110 is being operated.

The system 200 may be communicatively coupled (e.g., via a network, wireless network, cellular network, etc.) to a profile database containing information about the vehicle operator 105. The profile database may include a variety of information about the vehicle operator 105 such as, for example, age, routes traveled, average speed, frequently visited locations, medical conditions, length of time operating vehicles, etc.

The profile information may be retrieved from the profile database upon identifying the vehicle operator 105. The system 200 may identify the vehicle operator 105 using a variety of techniques. For example, the system 200 may receive a signal from a key fob used by the vehicle operator 105 in operating the vehicle 110. The key fob may be associated with the vehicle operator 105 and profile data may be selected for the vehicle operator 105 based on the association. In another example, a camera (e.g., in the vehicle 110, the mobile device 115, the HUD 102, etc.) may capture an image of the vehicle operator 105 and the image may be processed using facial recognition techniques to identify the vehicle operator 105 and profile data may be selected for the vehicle operator 105 from the profile database based on the facial recognition. In another example, a biometric sensor (e.g., in the vehicle 110, the mobile device 115, the HUD, etc.) may collect biometric data and may match the biometric to the vehicle operator 105 and may select the profile data from the profile database based on the match. In another example, the vehicle operator 105 may be identified based on a device (e.g., mobile device 115, etc.) paired (e.g., Bluetooth, etc.) with the vehicle 110. In some cases, multiple devices may be paired with the vehicle 110 and the vehicle operator 110 may be determined using various techniques such as, for example, matching data of a paired device to ownership data of the vehicle 110, receiving operator selection inputs from an infotainment system of the vehicle 110, etc.

The system 200 may receive the profile data for the vehicle operator 105 and may extract features from the profile data. The profile data may be evaluated using a model for the identified context to determine driver assistance notification modes. The determined driver assistance notification modes may be presented to the vehicle operator 105 operating the vehicle 110 in the identified context. The vehicle 110, mobile device 115, and HUD 120 may have a variety of driver assistance notification modes that may be used to notify the vehicle operator of hazards and other circumstance of which the vehicle operator 105 should be aware. For example, the vehicle 110 may have actuators that may cause the seat, steering wheel, or other component of the vehicle 110 to provide haptic feedback. The vehicle 110 may include a variety of display devices and audio devices that may be used to provide audio or visual notifications to be presented to the vehicle operator 105. The mobile device 115 and HUD 120 may include similar devices for presenting haptic, audio, and/or visual notifications to the vehicle operator 105.

The driver assistance notification modes may include presenting (or not presenting) various individual and/or combinations of feedback to the vehicle operator 105 based on the context in which the vehicle 110 is being operated and the profile data of the vehicle operator 105.

For example, the vehicle operator 105 may be an experienced operator (e.g., has traveled the current route several times, has been operating a vehicle for many years, etc.) may be operating the vehicle 110 in densely packed high speed traffic on a suburban freeway. The vehicle 110 may be equipped with lane departure and blind spot detection systems. Alerts may be delivered via both visual and haptic notifications based on the context (e.g., dense traffic, high speed, and suburban freeway), the experience of the vehicle operator 105, and the awareness level of the vehicle operator 105 (e.g., as determined from the sensor data from the HUD 120, etc.). In a typical case, notifications may be delivered to the HUD 120 and haptic and in-vehicle visual notifications may be suppressed. Over speed warnings may be mitigated if the majority of the surrounding traffic is traveling at a speed that between 0 and 10 miles over the posted speed limit for the location of the vehicle 110. Additional notification modalities may be used in the event the system 200 determines that the traffic conditions exceed a comfort level of the vehicle operator 105 (e.g., the context has changed such that the driver profile data no longer fits the context model, etc.). In this case, haptic feedback in the form of seat vibrations (e.g., left and right) may be re-enabled until the system 200 determines it is appropriate (e.g., the context has change such that the original context model again fits the profile information, etc.) to revert to the normal state having warned the driver that haptic feedback was being disabled.

In another example, the vehicle operator 105 may be an experienced operator with the vehicle 110 traveling a long distance on a sparsely populated highway. In this scenario, the HUD 120 may be used to deliver notifications of approaching vehicles to alert the vehicle operator 105 to things that might have been missed in the rear view or side view mirrors. These explicit notifications may compensate for driver fatigue and they may be indicated as early as possible based on input from cameras in the vehicle 110. In the event, the vehicle operator 105 starts to move into the path of a passing (e.g., oncoming) vehicle, the HUD 120 may use an audio system of the vehicle 110 to provide a spoken warning over the in-vehicle audio system. In the event the traffic conditions change to a more congested (e.g., urban, etc.) context, the HUD 120 in conjunction with the other driver assist systems (e.g., lane departure, collision avoidance, etc.) in the vehicle 110 may adapt to a mode mentioned above. In other words, the context has changed, and thus, the notification modes have changed to reflect the updated context.

In another example, the vehicle operator 105 may be a senior driver traveling with GPS assist to a new destination in an urban setting. The context may include that that the destination is not a location the vehicle operator 105 has visited before. In this case, visual notifications may be directed to the HUD 120 and audio notifications may be directed to the in-vehicle audio system. Notifications may be delivered more frequently and may be presented more quickly to provide the vehicle operator 105 with additional time to take corrective action (e.g., turn, change lanes, slow down, etc.).

In another example, the vehicle operator 105 may be a novice operator traveling on freeway moving at the speed limit. The system 200 may use cameras and proximity sensors included in the vehicle 110 to display (e.g., using the HUD 120, a display of the vehicle 110, etc.) the relative location of the vehicles on the road. This may reduce the anxiety faced by a novice operator that may not be familiar with the spatial relationship between the vehicle 110 and other vehicles operating nearby. The modalities of notifications may be adapted so as to enhance the awareness of risk of the vehicle operator 105 based on measure of the awareness of the vehicle operator 105 (e.g., head direction, gaze tracking, etc.). For example, a haptic sensor in a seat of the vehicle 110 may be caused to vibrate at a location that corresponds to the right hip of the vehicle operator 105 when the vehicle operator 105 is drifting right and looking left or straight ahead.

In another example, the vehicle operator 105 may have a great degree of familiarity with the location where the vehicle 110 is traveling. For example, the vehicle 110 may be at a drive-up Automated Teller Machine (ATM) or a drive through window of a fast food restaurant which the vehicle operator 105 has visited many times. The system 200 may learn (e.g., using machine learning, etc.) that the vehicle operator 105 is accustomed to the narrow lane width and height at these locations and may not alert the vehicle operator 105 to nearby objects as quickly as the visits increase. When the vehicle 110 is carrying a load either in a rack on top or rack attached to a trailer hitch the system 200 may transmit alerts to the vehicle 110 and HUD 120 to progressively alert the driver to the environment. For example, upon approach to an ATM with low clearance, a visual alert may be displayed on the HUD 120 to remind the vehicle operator 105 that there is cargo on top of the vehicle 110. If the vehicle operator 105 continues to approach the ATM, a haptic alert may be delivered (e.g., to a steering wheel of the vehicle 110, etc.) followed by an audible alert (e.g., transmitted to an in-vehicle audio system, etc.) as the vehicle 110 approaches the obstacle.

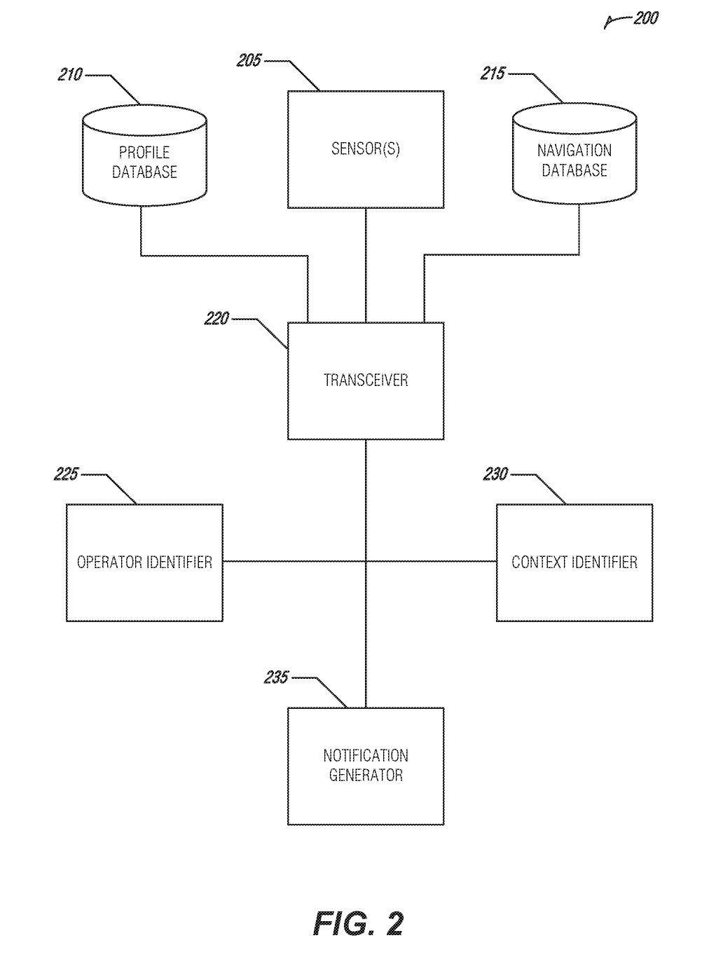

FIG. 2 is a block diagram of an example of a system 200 for context derived driver assistance, according to an embodiment. The system 200 may provide the features described in FIG. 1. The system 200 may include sensor(s) 205, a profile database 210, and a navigation database 215 that are communicatively coupled (e.g., via wireless network, shortwave radio, cellular network, etc.) to a transceiver 220. The transceiver 220 may be communicatively coupled (e.g., via network, shared bus, etc.) to an operator identifier 225, context identifier 230, and a notification generator 235.

The sensor(s) 205 may include a variety of sensors including, for example, a camera, global positioning system (GPS) sensor, accelerometer, proximity sensor, gyroscope, magnetometer, microphone, etc. The sensor(s) 205 may be included in a vehicle (e.g., vehicle 110 as described in FIG. 1, etc.), a mobile device (e.g., mobile device 115 as described in FIG. 1), heads-up display (HUD) (e.g., HUD 120 as described in FIG. 1), etc. The sensors may collect data corresponding to the context in which the vehicle is operated as well as data corresponding to a vehicle operator (e.g., vehicle operator 105 as described in FIG. 1).

The profile database 210 may contain a variety of information about the vehicle operator such as, for example, age, routes traveled, locations visited, how long the vehicle operator has operated vehicles, preferences, etc. The profile database 210 may be used by the system 200 in determining modes for delivering driver assistance notifications to the vehicle operator. For example, the profile information may be used to determine an experience level of the vehicle operator and modes of notification may be selected in part based on the experience level of the vehicle operator.

The navigation database 215 may contain a variety of information such as, for example, route information, traffic information for a route, location data, point of interest information, etc. The navigation database 215 may be used to determine a context in which the vehicle is being operated. For example, the navigation information may be used to identify traffic volume along a route and/or at a location where the vehicle is traveling.

The transceiver 220 may process and direct incoming and outgoing data. For example, the transceiver 220 may receive data from the profile database 210, sensor(s) 205, and navigation database 215 and may forward the data on to other components of the system 200. In another example, the transceiver 220 may receive data from the notification generator 235 (e.g., modes of notification, actuator activation data, audio data, visual data, etc.) and may forward the data to components of the vehicle, mobile device, and/or HUD.

The operator identifier 225 may determine an identity of a vehicle operator of the vehicle. In an example, a signal may be received from a key fob used in operating the vehicle and the identity of the vehicle operator may be determined based on an association between the key fob and the vehicle operator. In another example, an image of an operating position of the vehicle may be captured. The image may be evaluated (e.g., using facial recognition techniques, etc.) to identify an object in the image and the identity of the vehicle operator may be based on the object in the image matching a face of the vehicle operator. In another example, biometric data may be received from a biometric sensor of the vehicle and the identity of the vehicle operator may be based on a match between the received biometric data and biometric data of the vehicle operator. In another example, the vehicle operator may be identified based on a device (e.g., mobile device 115 as described in FIG. 1, etc.) paired (e.g., Bluetooth, etc.) with the vehicle. In some cases, multiple devices may be paired with the vehicle and the vehicle operator may be determined using various techniques such as, for example, matching data of a paired device to ownership data of the vehicle, receiving operator selection inputs from an infotainment system of the vehicle, etc. The identity of the vehicle operator may be used to retrieve profile data for the vehicle operator from the profile database 210.

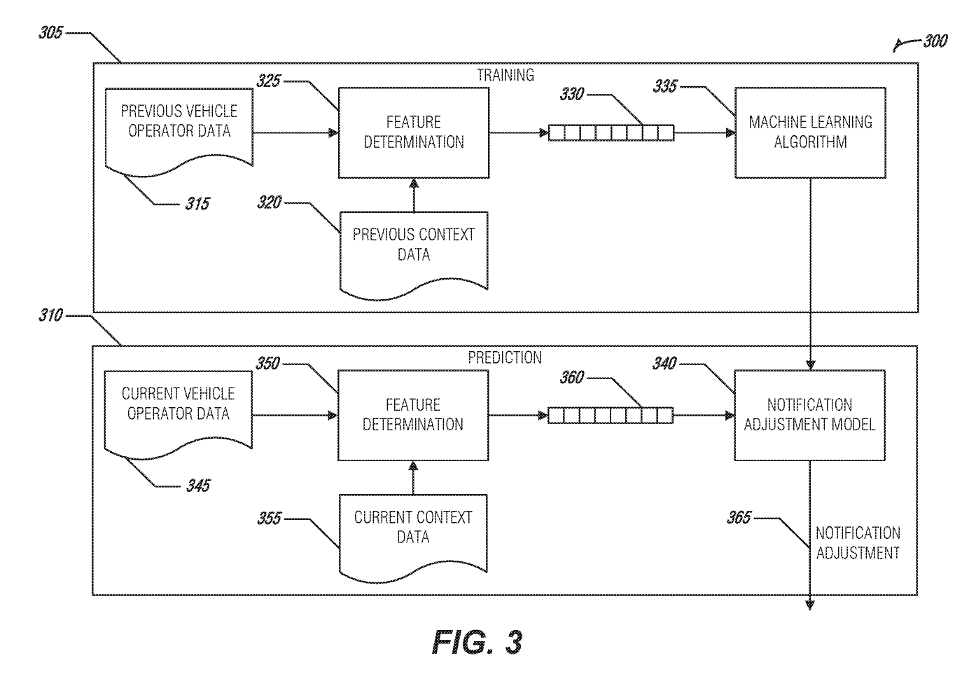

The context identifier 230 may determine an operating context of the vehicle. In an example, a set of sensor data may be received (e.g., collected from the sensor(s) 205 and received by the transceiver 220) from the vehicle and the operating context of the vehicle may be determined using the set of sensor data. In an example, the set of sensor data may include data from a GPS sensor, an accelerometer, a gyroscope, a magnetometer, a collision detection system, a lane departure system, a speed sensor, or a weather sensor. For example, images, speed, proximity, and GPS data may be received and evaluated to determine the operating context. In some examples, machine learning techniques such as those described in FIG. 3 may be used with the sensor data to determine the operating context. For example, location data, traffic volume data, and speed data may be extracted from the sensor data to determine that the operating context is a suburban freeway with light traffic at freeway speed (e.g., 65 mile per hour, etc.).

The notification generator 235 may adaptively adjust a notification concerning the operating context based on the identity of the vehicle operator. For example, the vehicle, mobile device, and HUD may include components that may be actuated to provide audio, visual, and/or haptic feedback to the vehicle operator to prompt the vehicle operator to take corrective action (or avoid taking an action). The notification generator 235 may adjust the notifications provided to the vehicle operator based on the vehicle operator's identity and the operating context.

In some examples, the notification may be prepared in a first state for presentation to the vehicle operator. The first state may include a first mode. For example, the notification may be prepared for presentation as a vibration in a seat of the vehicle. The notification may be presented in a second state to the vehicle operator based on a change in the operating context. The second state may include a second mode. For example, the traffic volume near where the vehicle is traveling may have reduced and the notification may be adjusted to a display in a side mirror of the vehicle. In an example, the first mode and the second mode may include haptic, visual, or audio output. In an example, the first mode and the second mode may include output to a heads up display, a steering wheel of the vehicle, a seat of the vehicle, a lane departure warning system of the vehicle, a collision detection system of the vehicle, an audio device of the vehicle, or a display of the vehicle. For example, either or both of the first and second modes may include activation of a haptic feedback feature, audio feedback feature, or visual feedback feature of the vehicle, mobile device, or the HUD.

In another example, the second mode may include an adjustment to a notification intensity level of the first mode. For example, the first mode may include a light vibration of a seat of the vehicle and it may be determined that traffic has increased near the vehicle and the second mode may modify the first mode to be a heavy vibration in the seat of the vehicle based on the determination of an increase in traffic volume. In another example, the second mode may include disablement of the first mode. For example, the vehicle may have moved into a rural area with very light traffic and vibration of the seat may be disabled.

In another example, a direction of gaze of the vehicle operator may be determined and the notification may be presented in the second state to the vehicle operator based on the direction of gaze. In another example, a load on the vehicle (e.g., items on top of the vehicle, item on a load hitch of the vehicle, etc.) may be determined and the notification may be presented in the second state to the vehicle operator based on the load on the vehicle.

In some examples, the notification may be prepared in a first state for presentation to the vehicle operator and the notification may be presented in a second state to the vehicle operator based on the identity of the vehicle operator. The first state may include a first mode and the second state may include a second mode. In an example, the first mode and the second mode may include haptic, visual, or audio output. In an example, the second mode may include an adjustment to a notification intensity level of the first mode. In an example, the second mode may include disablement of the first mode.

In some examples, a familiarity of the vehicle operator with a location of the vehicle (e.g., how many times has the vehicle operator visited the location as indicated in the profile database, etc.) may be determined using the identity of the vehicle operator and the notification may be presented in a second state to the vehicle operator based on the familiarity of the vehicle operator with the location of the vehicle. For example, the vehicle operator may have visited a drive-up teller window at a bank a number of times and the notification may be prepared for presentation to the vehicle operator as an audible alert from an in-vehicle audio system of the vehicle but the audible alert may be disabled and a visual indicator may be presented in a dash display cluster of the vehicle.

In some examples, a vehicle operation experience level of the operator may be determined using the identity of the vehicle operator. For example, the transceiver 220 may receive profile data for the vehicle operator including how many years the vehicle operator has been operating vehicles and it may be determined the vehicle operator has a high level of experience because the vehicle operator has been operating vehicles for 10 years. The notification may be presented in the second state to the vehicle operator based on the vehicle operation experience level of the vehicle operator. For example, the notification may be prepared for presentation as a vibration of a steering wheel of the vehicle and based on the vehicle operator having a high operation experience level, the notification may be presented as a visual indicator on the HUD.

FIG. 3 illustrates an example machine learning system 300 for notification adjustment for context derived driver assistance, according to some embodiments. The machine learning system 300 utilizes a training component 305 and a prediction component 310. Training component 305 feeds previous vehicle operator data 315 and previous context data 320 into feature determination component 325 which determines one or more features 330 from the data. Features 330 are a subset of the information input and is information determined to be predictive of notification adjustments likely to provide assistance to a vehicle operator operating a vehicle in an operating context. Examples include one or more of: age of the vehicle operator, how long the vehicle operator has been operating vehicles, routes visited by a vehicle operator, traffic conditions (volume, speed, etc.), traffic conditions on routes previously traveled by the vehicle operator, weather conditions, vehicle operator attentiveness, etc.

The machine learning algorithm 335 produces a notification adjustment model 340 based upon the features and feedback associated with those features. For example, the features associated with previous vehicle operators in various operating contexts are used as a set of training data. As noted above, the notification adjustment model 340 may be for the entire system, or may be built specific for each vehicle operator, each operating context, or vehicle operator and operating context pair.

In the prediction component 310, the current vehicle operator data 345 may be input to the feature determination component 350. Similarly the current context data 355 is also input to the feature determination component 350. Feature determination component 350 may determine the same set of features or a different set of features as feature determination component 325. In some examples, feature determination component 350 and 325 are the same component. Feature determination component 350 produces features 360, which are input into the notification adjustment model 340 to generate a notification adjustment prediction 365. The training component 305 may operate in an offline manner to train the notification adjustment model 340. The prediction component 310, however, may be designed to operate in an online manner as the current vehicle operator is evaluated in the current context.

It should be noted that the notification adjustment model 340 may be periodically updated via additional training and/or user feedback. The user feedback may be either feedback from users giving explicit feedback (e.g., responses to questions about whether the notification adjustment was accurate, etc.).

The machine learning algorithm 335 may be selected from among many different potential supervised or unsupervised machine learning algorithms. Examples of supervised learning algorithms include artificial neural networks. Bayesian networks, instance-based learning, support vector machines, decision trees (e.g., Iterative Dichotomiser 3, C4.5, Classification and Regression Tree (CART), Chi-squared Automatic Interaction Detector (CHAID), and the like), random forests, linear classifiers, quadratic classifiers, k-nearest neighbor, linear regression, sensory adaptation, neural adaptation, and hidden Markov models. Examples of unsupervised learning algorithms include expectation-maximization algorithms, vector quantization, and information bottleneck method. In an example embodiment, a multi-class logistical regression model is used.

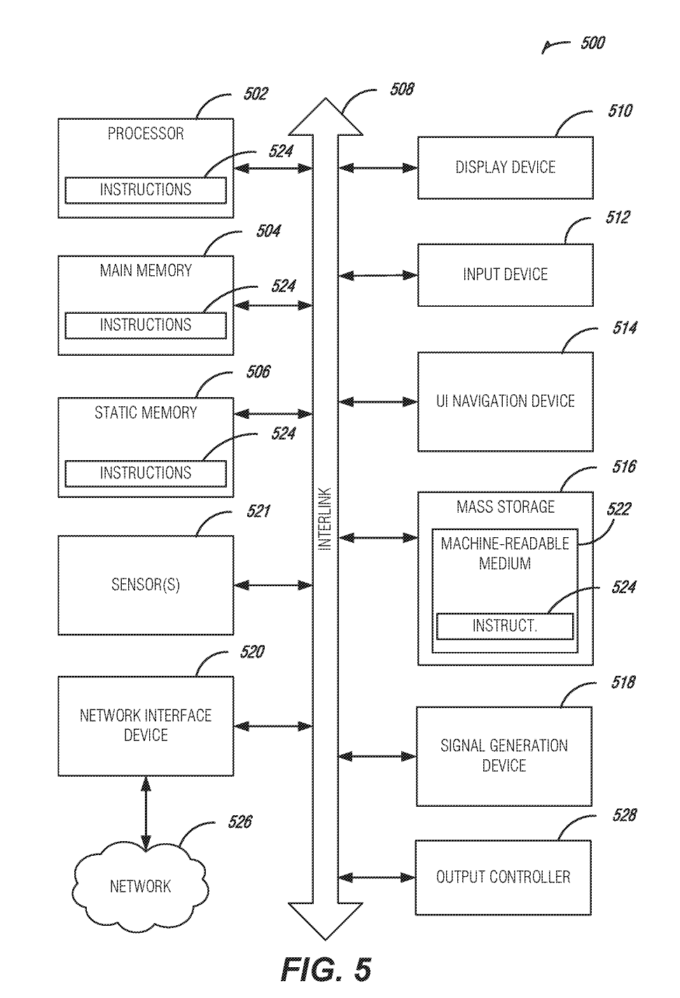

The system 200 and machine learning system 300 may be implemented on one or more computing devices, such as machine 500 of FIG. 5. As such, some of the components of FIGS. 2 & 5 may communicate with each other via inter-process communication and other local communications techniques (e.g., shared memory, pipes, buffers, queues). In other examples, the components of FIGS. 2 & 5 may be parts of different services or systems and thus the components may communicate with each other through a computer network using computer networking protocols.

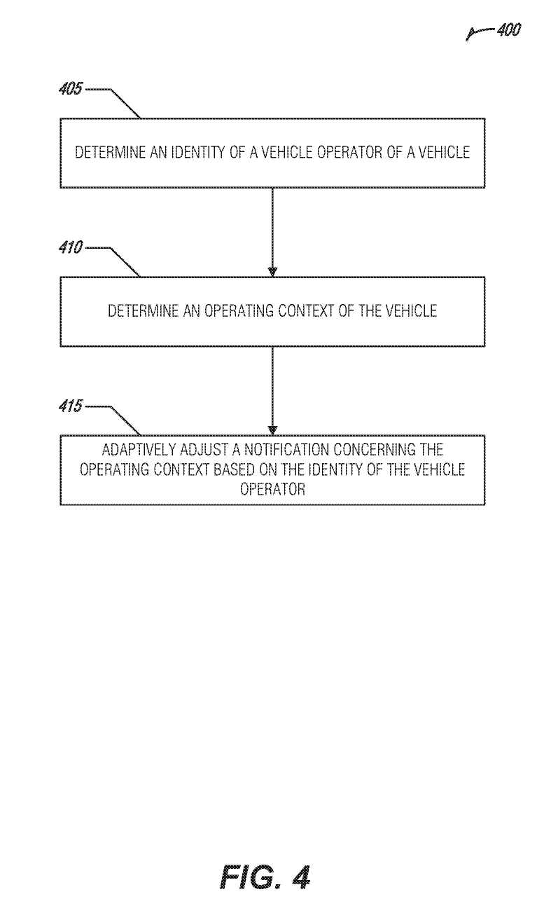

FIG. 4 illustrates an example of a method 400 for context derived driver assistance, according to an embodiment. The method 400 may provide the functionality described in FIGS. 1-3.

At operation 405, an identity of a vehicle operator may be determined. In an example, a signal may be received from a key fob used in operating the vehicle and the identity of the vehicle operator may be determined based on an association between the key fob and the vehicle operator. In another example, an image may be captured of an operating position of the vehicle. The image may be evaluated to identify an object in the image and the identity of the vehicle operator may be determined based on the object in the image matching a face of the vehicle operator. In another example, biometric data may be received from a biometric sensor of the vehicle and the identity of the vehicle operator may be identified based on a match between the received biometric data and biometric data of the vehicle operator.

At operation 410, an operating context of the vehicle may be determined. In an example, a set of sensor data may be received from the vehicle and the operating context may be determined using the set of sensor data. In an example, the set of sensor data may include data from a global positioning sensor, an accelerometer, a gyroscope, a magnetometer, a collision detection system, a lane departure detection system, a speed sensor, or a weather sensor.

At operation 415, a notification concerning the operating context may be adaptively adjusted based on the identity of the vehicle operator. In an example, the notification may be prepared in a first state for presentation to the vehicle operator and the notification may be presented in a second state to the vehicle operator based on a change in the operating context. In an example, the first state may include a first mode. In an example, the second state may include a second mode. In an example, the second mode may include an adjustment to a notification intensity level of the first mode. In another example, the second mode may include disablement of the first mode. In some examples, either or both of the first mode and/or the second mode may include haptic, visual, or audio output. In an example, either or both of the first mode and/or the second mode may include output to a heads up display, a steering wheel of the vehicle, a seat of the vehicle, a lane departure warning system of the vehicle, a collision detection system of the vehicle, an audio device of the vehicle, or a display of the vehicle.

In some examples, a direction of gaze may of the vehicle operator may be determined and the notification may be presented in the second state to the vehicle operator based on the direction of gaze. In some examples, a load on the vehicle may be determined and the notification may be presented in the second state to the vehicle operator based on the load on the vehicle.

In some examples, the notification may be prepared in a first state for presentation to the vehicle operator and the notification in a second state may be presented to the vehicle operator based on the identity of the vehicle operator. In an example, a familiarity of the vehicle operator with a location of the vehicle may be determined using the identity of the vehicle operator and the notification may be presented in the second state to the vehicle operator based on the familiarity of the vehicle operator with the location of the vehicle. In another example, a vehicle operation experience level of the vehicle operator may be determined using the identity of the vehicle operator and the notification may be presented in the second state to the vehicle operator based on the vehicle operation experience level of the vehicle operator. The first state may include a first mode. The second state may include a second mode. In an example, the second mode may include an adjustment to a notification intensity level of the first mode. In another example, the second mode may include disablement of the first mode. In some examples, either or both of the first mode and/or the second mode may include haptic, visual, or audio output.

FIG. 5 illustrates a block diagram of an example machine 500 upon which any one or more of the techniques (e.g., methodologies) discussed herein may perform. In alternative embodiments, the machine 500 may operate as a standalone device or may be connected (e.g., networked) to other machines. In a networked deployment, the machine 500 may operate in the capacity of a server machine, a client machine, or both in server-client network environments. In an example, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, in-car infotainment device, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Examples, as described herein, may include, or may operate by, logic or a number of components, or mechanisms. Circuit sets are a collection of circuits implemented in tangible entities that include hardware (e.g., simple circuits, gates, logic, etc.). Circuit set membership may be flexible over time and underlying hardware variability. Circuit sets include members that may, alone or in combination, perform specified operations when operating. In an example, hardware of the circuit set may be immutably designed to carry out a specific operation (e.g., hardwired). In an example, the hardware of the circuit set may include variably connected physical components (e.g., execution units, transistors, simple circuits, etc.) including a computer readable medium physically modified (e.g., magnetically, electrically, moveable placement of invariant massed particles, etc.) to encode instructions of the specific operation. In connecting the physical components, the underlying electrical properties of a hardware constituent are changed, for example, from an insulator to a conductor or vice versa. The instructions enable embedded hardware (e.g., the execution units or a loading mechanism) to create members of the circuit set in hardware via the variable connections to carry out portions of the specific operation when in operation. Accordingly, the computer readable medium is communicatively coupled to the other components of the circuit set member when the device is operating. In an example, any of the physical components may be used in more than one member of more than one circuit set. For example, under operation, execution units may be used in a first circuit of a first circuit set at one point in time and reused by a second circuit in the first circuit set, or by a third circuit in a second circuit set at a different time.

Machine (e.g., computer system) 500 may include a hardware processor 502 (e.g., a central processing unit (CPU), a graphics processing unit (GPU), a hardware processor core, or any combination thereof), a main memory 504 and a static memory 506, some or all of which may communicate with each other via an interlink (e.g., bus) 508. The machine 500 may further include a display unit 510, an alphanumeric input device 512 (e.g., a keyboard), and a user interface (UI) navigation device 514 (e.g., a mouse). In an example, the display unit 510, input device 512 and UI navigation device 514 may be a touch screen display. The machine 500 may additionally include a storage device (e.g., drive unit) 516, a signal generation device 518 (e.g., a speaker), a network interface device 520, and one or more sensors 521, such as a global positioning system (GPS) sensor, compass, accelerometer, or other sensor. The machine 500 may include an output controller 528, such as a serial (e.g., universal serial bus (USB), parallel or other wired or wireless (e.g., infrared (IR), near field communication (NFC), etc.) connection to communicate or control one or more peripheral devices (e.g., a printer, card reader, etc.).

The storage device 516 may include a machine readable medium 522 on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the machine 500. In an example, one or any combination of the hardware processor 502, the main memory 504, the static memory 506, or the storage device 516 may constitute machine readable media.

While the machine readable medium 522 is illustrated as a single medium, the term “machine readable medium” may include a single medium or multiple media (e.g., a centralized or distributed database, and/or associated caches and servers) configured to store the one or more instructions 524.

The term “machine readable medium” may include any medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Non-limiting machine readable medium examples may include solid-state memories, and optical and magnetic media. In an example, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM). Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, Bluetooth low energy, OBD II, CAN bus, among others. In an example, the network interface device 520 may include one or more physical jacks (e.g., Ethernet, coaxial, or phone jacks) or one or more antennas to connect to the communications network 526. In an example, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software.

### Additional Notes & Examples

Example 1 is a system for context derived driver assistance, the system comprising: at least one processor; and a memory including instructions that, when executed by the at least one processor, cause the at least one processor to perform operations to: determine an identity of a vehicle operator of a vehicle; determine an operating context of the vehicle; and adaptively adjust a notification concerning the operating context based on the identity of the vehicle operator.

In Example 2, the subject matter of Example 1 optionally includes the operations to determine the identity of the vehicle operator comprising operations to: receive a signal from a key fob used in operating the vehicle; and determine the identity of the vehicle operator based on an association between the key fob and the vehicle operator.

In Example 3, the subject matter of any one or more of Examples 1-2 optionally include the operations to determine the identity of the vehicle operator comprising operations to: capture an image of an operating position of the vehicle; evaluate the image to identify an object in the image; and determine the identity of the vehicle operator based on the object in the image matching a face of the vehicle operator.

In Example 4, the subject matter of any one or more of Examples 1-3 optionally include the operations to determine the identity of the vehicle operator comprising operations to: receive biometric data from a biometric sensor of the vehicle; and determine the identity of the vehicle operator based on a match between the received biometric data and biometric data of the vehicle operator.

In Example 5, the subject matter of any one or more of Examples 1-4 optionally include the operations to determine the operating context of the vehicle comprising operations to: receive a set of sensor data from the vehicle; and determine the operating context of the vehicle using the set of sensor data.

In Example 6, the subject matter of Example 5 optionally includes wherein the set of sensor data includes data from a global positioning sensor, an accelerometer, a gyroscope, a magnetometer, a collision detection system, a lane departure detection system a speed sensor, or a weather sensor.

In Example 7, the subject matter of any one or more of Examples 1-6 optionally include the operations to adaptively adjust the notification comprising operations to: prepare the notification in a first state for presentation to the vehicle operator, the first state including a first mode; and present the notification in a second state to the vehicle operator based on a change in the operating context, the second state including a second mode.

In Example 8, the subject matter of Example 7 optionally includes wherein the first mode and second mode includes haptic, visual, or audio output.

In Example 9, the subject matter of any one or more of Examples 7-8 optionally include wherein the second mode includes an adjustment to a notification intensity level of the first mode.

In Example 10, the subject matter of any one or more of Examples 7-9 optionally include wherein the second mode includes disablement of the first mode.

In Example 11, the subject matter of any one or more of Examples 7-10 optionally include wherein the first mode and the second mode includes output to a heads up display, a steering wheel of the vehicle, a seat of the vehicle, a lane departure warning system of the vehicle, a collision detection system of the vehicle, an audio device of the vehicle, or a display of the vehicle.

In Example 12, the subject matter of any one or more of Examples 7-11 optionally include the operations to present the notification in the second state to the vehicle operator based on a change in the operating context comprising operations to: determine a direction of gaze of the vehicle operator; and present the notification in the second state to the vehicle operator based on the direction of gaze.

In Example 13, the subject matter of any one or more of Examples 7-12 optionally include the operations to present the notification in the second state to the vehicle operator based on a change in the operating context comprising operations to: determine a load on the vehicle; and present the notification in the second state to the vehicle operator based on the load on the vehicle.

In Example 14, the subject matter of any one or more of Examples 1-13 optionally include the operations to adaptively adjust the notification comprising operations to: prepare the notification in a first state for presentation to the vehicle operator, the first state including a first mode; and present the notification in a second state to the vehicle operator based on the identity of the vehicle operator, the second state including a second mode.

In Example 15, the subject matter of Example 14 optionally includes the operations to present the notification in the second state to the vehicle operator based on the identity of the vehicle operator comprising operations to: determine a familiarity of the vehicle operator with a location of the vehicle using the identity of the vehicle operator; and present the notification in the second state to the vehicle operator based on the familiarity of the vehicle operator with the location of the vehicle.

In Example 16, the subject matter of any one or more of Examples 14-15 optionally include the operations to present the notification in the second state to the vehicle operator based on the identity of the vehicle operator comprising operations to: determine a vehicle operation experience level of the vehicle operator using the identity of the vehicle operator; and present the notification in the second state to the vehicle operator based on the vehicle operation experience level of the vehicle operator.

In Example 17, the subject matter of any one or more of Examples 14-16 optionally include wherein the first mode and second mode includes haptic, visual, or audio output.

In Example 18, the subject matter of any one or more of Examples 14-17 optionally include wherein the second mode includes an adjustment to a notification intensity level of the first mode.

In Example 19, the subject matter of any one or more of Examples 14-18 optionally include wherein the second mode includes disablement of the first mode.

In Example 20, the subject matter of any one or more of Examples 1-19 optionally include wherein the operating context includes a set of environmental attributes that define an environment in which the vehicle is operating and a set of operator attributes that define a current state of the vehicle operator.

In Example 21, the subject matter of any one or more of Examples 15-20 optionally include the operations to determine the familiarity of the vehicle operator further comprising operations to: analyze operator profile data from a profile database to determine how long the vehicle operator has been operating vehicles.

Example 22 is at least one machine readable medium including instructions for context derived driver assistance that, when executed by a machine, cause the machine to perform operations to: determine an identity of a vehicle operator of a vehicle; determine an operating context of the vehicle; and adaptively adjust a notification concerning the operating context based on the identity of the vehicle operator.

In Example 23, the subject matter of Example 22 optionally includes the operations to determine the identity of the vehicle operator comprising operations to: receive a signal from a key fob used in operating the vehicle; and determine the identity of the vehicle operator based on an association between the key fob and the vehicle operator.

In Example 24, the subject matter of any one or more of Examples 22-23 optionally include the operations to determine the identity of the vehicle operator comprising operations to: capture an image of an operating position of the vehicle; evaluate the image to identify an object in the image; and determine the identity of the vehicle operator based on the object in the image matching a face of the vehicle operator.

In Example 25, the subject matter of any one or more of Examples 22-24 optionally include the operations to determine the identity of the vehicle operator comprising operations to: receive biometric data from a biometric sensor of the vehicle; and determine the identity of the vehicle operator based on a match between the received biometric data and biometric data of the vehicle operator.

In Example 26, the subject matter of any one or more of Examples 22-25 optionally include the operations to determine the operating context of the vehicle comprising operations to: receive a set of sensor data from the vehicle; and determine the operating context of the vehicle using the set of sensor data.

In Example 27, the subject matter of Example 26 optionally includes wherein the set of sensor data includes data from a global positioning sensor, an accelerometer, a gyroscope, a magnetometer, a collision detection system, a lane departure detection system, a speed sensor, or a weather sensor.

In Example 28, the subject matter of any one or more of Examples 22-27 optionally include the operations to adaptively adjust the notification comprising operations to: prepare the notification in a first state for presentation to the vehicle operator, the first state including a first mode; and present the notification in a second state to the vehicle operator based on a change in the operating context, the second state including a second mode.

In Example 29, the subject matter of Example 28 optionally includes wherein the first mode and second mode includes haptic, visual, or audio output.

In Example 30, the subject matter of any one or more of Examples 28-29 optionally include wherein the second mode includes an adjustment to a notification intensity level of the first mode.

In Example 31, the subject matter of any one or more of Examples 28-30 optionally include wherein the second mode includes disablement of the first mode.

In Example 32, the subject matter of any one or more of Examples 28-31 optionally include wherein the first mode and the second mode includes output to a heads up display, a steering wheel of the vehicle, a seat of the vehicle, a lane departure warning system of the vehicle, a collision detection system of the vehicle, an audio device of the vehicle, or a display of the vehicle.

In Example 33, the subject matter of any one or more of Examples 28-32 optionally include the operations to present the notification in the second state to the vehicle operator based on a change in the operating context comprising operations to: determine a direction of gaze of the vehicle operator; and present the notification in the second state to the vehicle operator based on the direction of gaze.

In Example 34, the subject matter of any one or more of Examples 28-33 optionally include the operations to present the notification in the second state to the vehicle operator based on a change in the operating context comprising operations to: determine a load on the vehicle; and present the notification in the second state to the vehicle operator based on the load on the vehicle.

In Example 35, the subject matter of any one or more of Examples 22-34 optionally include the operations to adaptively adjust the notification comprising operations to: prepare the notification in a first state for presentation to the vehicle operator, the first state including a first mode; and present the notification in a second state to the vehicle operator based on the identity of the vehicle operator, the second state including a second mode.

In Example 36, the subject matter of Example 35 optionally includes the operations to present the notification in the second state to the vehicle operator based on the identity of the vehicle operator comprising operations to: determine a familiarity of the vehicle operator with a location of the vehicle using the identity of the vehicle operator; and present the notification in the second state to the vehicle operator based on the familiarity of the vehicle operator with the location of the vehicle.

In Example 37, the subject matter of any one or more of Examples 35-36 optionally include the operations to present the notification in the second state to the vehicle operator based on the identity of the vehicle operator comprising operations to: determine a vehicle operation experience level of the vehicle operator using the identity of the vehicle operator; and present the notification in the second state to the vehicle operator based on the vehicle operation experience level of the vehicle operator.

In Example 38, the subject matter of any one or more of Examples 35-37 optionally include wherein the first mode and second mode includes haptic, visual, or audio output.

In Example 39, the subject matter of any one or more of Examples 35-38 optionally include wherein the second mode includes an adjustment to a notification intensity level of the first mode.

In Example 40, the subject matter of any one or more of Examples 36-39 optionally include wherein the second mode includes disablement of the first mode.

In Example 41, the subject matter of any one or more of Examples 22-40 optionally include wherein the operating context includes a set of environmental attributes that define an environment in which the vehicle is operating and a set of operator attributes that define a current state of the vehicle operator.

In Example 42, the subject matter of any one or more of Examples 36-41 optionally include the operations to determine the familiarity of the vehicle operator comprising operations to: analyze operator profile data from a profile database to determine how long the vehicle operator has been operating vehicles.

Example 43 is a method for context derived driver assistance, the method comprising: determining an identity of a vehicle operator of a vehicle using profile data from a profile database; determining an operating context of the vehicle using environmental data, the environmental data including sensor data; and adaptively adjusting an electronic notification concerning the operating context based on the identity of the vehicle operator.

In Example 44, the subject matter of Example 43 optionally includes wherein determining the identity of the vehicle operator comprises: receiving a signal from a key fob used in operating the vehicle; and determining the identity of the vehicle operator based on an association between the key fob and the vehicle operator.

In Example 45, the subject matter of any one or more of Examples 43-44 optionally include wherein determining the identity of the vehicle operator comprises: capturing an image of an operating position of the vehicle; evaluating the image to identify an object in the image; and determining the identity of the vehicle operator based on the object in the image matching a face of the vehicle operator.

In Example 46, the subject matter of any one or more of Examples 43-45 optionally include wherein determining the identity of the vehicle operator comprises: receiving biometric data from a biometric sensor of the vehicle; and determining the identity of the vehicle operator based on a match between the received biometric data and biometric data of the vehicle operator.

In Example 47, the subject matter of any one or more of Examples 43-46 optionally include wherein determining the operating context of the vehicle comprises: receiving a set of sensor data from the vehicle; and determining the operating context of the vehicle using the set of sensor data.

In Example 48, the subject matter of Example 47 optionally includes wherein the set of sensor data includes data from a global positioning sensor, an accelerometer, a gyroscope, a magnetometer, a collision detection system, a lane departure detection system, a speed sensor, or a weather sensor.

In Example 49, the subject matter of any one or more of Examples 43-48 optionally include wherein adaptively adjusting the electronic notification comprises: preparing the electronic notification in a first state for presentation to the vehicle operator, the first state including a first mode; and presenting the electronic notification in a second state to the vehicle operator based on a change in the operating context, the second state including a second mode.

In Example 50, the subject matter of Example 49 optionally includes wherein the first mode and second mode includes haptic, visual, or audio output.

In Example 51, the subject matter of any one or more of Examples 49-50 optionally include wherein the second mode includes an adjustment to a notification intensity level of the first mode.

In Example 52, the subject matter of any one or more of Examples 49-51 optionally include wherein the second mode includes disablement of the first mode.

In Example 53, the subject matter of any one or more of Examples 49-52 optionally include wherein the first mode and the second mode includes output to a heads up display, a steering wheel of the vehicle, a seat of the vehicle, a lane departure warning system of the vehicle, a collision detection system of the vehicle, an audio device of the vehicle, or a display of the vehicle.

In Example 54, the subject matter of any one or more of Examples 49-53 optionally include wherein presenting the electronic notification in the second state to the vehicle operator based on a change in the operating context comprises: determining a direction of gaze of the vehicle operator; and presenting the electronic notification in the second state to the vehicle operator based on the direction of gaze.

In Example 55, the subject matter of any one or more of Examples 49-54 optionally include wherein presenting the electronic notification in the second state to the vehicle operator based on a change in the operating context comprises: determining a load on the vehicle; and presenting the electronic notification in the second state to the vehicle operator based on the load on the vehicle.

In Example 56, the subject matter of any one or more of Examples 43-55 optionally include wherein adaptively adjusting the electronic notification comprises: preparing the electronic notification in a first state for presentation to the vehicle operator, the first state including a first mode; and presenting the electronic notification in a second state to the vehicle operator based on the identity of the vehicle operator, the second state including a second mode.

In Example 57, the subject matter of Example 56 optionally includes wherein presenting the electronic notification in the second state to the vehicle operator based on the identity of the vehicle operator comprises: determining a familiarity of the vehicle operator with a location of the vehicle using the identity of the vehicle operator; and presenting the electronic notification in the second state to the vehicle operator based on the familiarity of the vehicle operator with the location of the vehicle.

In Example 58, the subject matter of any one or more of Examples 56-57 optionally include wherein presenting the electronic notification in the second state to the vehicle operator based on the identity of the vehicle operator comprises: determining a vehicle operation experience level of the vehicle operator using the identity of the vehicle operator, and presenting the electronic notification in the second state to the vehicle operator based on the vehicle operation experience level of the vehicle operator.

In Example 59, the subject matter of any one or more of Examples 56-58 optionally include wherein the first mode and second mode includes haptic, visual, or audio output.

In Example 60, the subject matter of any one or more of Examples 56-59 optionally include wherein the second mode includes an adjustment to a notification intensity level of the first mode.

In Example 61, the subject matter of any one or more of Examples 56-60 optionally include wherein the second mode includes disablement of the first mode.

In Example 62, the subject matter of any one or more of Examples 43-61 optionally include wherein the operating context includes a set of environmental attributes that define an environment in which the vehicle is operating and a set of operator attributes that define a current state of the vehicle operator.

In Example 63, the subject matter of any one or more of Examples 57-62 optionally include wherein determining the familiarity of the vehicle operator comprises analyzing operator profile data from a profile database to determine how long the vehicle operator has been operating vehicles.

Example 64 is a system to implement context derived driver assistance, the system comprising means to perform any method of Examples 43-63.

Example 65 is at least one machine readable medium to implement context derived driver assistance, the at least one machine readable medium including instructions that, when executed by a machine, cause the machine to perform any method of Examples 43-63.

Example 66 is a system for context derived driver assistance, the system comprising: means for determining an identity of a vehicle operator of a vehicle; means for determining an operating context of the vehicle; and means for adaptively adjusting a notification concerning the operating context based on the identity of the vehicle operator.

In Example 67, the subject matter of Example 66 optionally includes wherein determining the identity of the vehicle operator comprises: means for receiving a signal from a key fob used in operating the vehicle; and means for determining the identity of the vehicle operator based on an association between the key fob and the vehicle operator.

In Example 68, the subject matter of any one or more of Examples 66-67 optionally include wherein determining the identity of the vehicle operator comprises: means for capturing an image of an operating position of the vehicle; means for evaluating the image to identify an object in the image; and means for determining the identity of the vehicle operator based on the object in the image matching a face of the vehicle operator.

In Example 69, the subject matter of any one or more of Examples 66-68 optionally include wherein determining the identity of the vehicle operator comprises: means for receiving biometric data from a biometric sensor of the vehicle; and means for determining the identity of the vehicle operator based on a match between the received biometric data and biometric data of the vehicle operator.

In Example 70, the subject matter of any one or more of Examples 66-69 optionally include wherein determining the operating context of the vehicle comprises: means for receiving a set of sensor data from the vehicle; and means for determining the operating context of the vehicle using the set of sensor data.

In Example 71, the subject matter of Example 70 optionally includes wherein the set of sensor data includes data from a global positioning sensor, an accelerometer, a gyroscope, a magnetometer, a collision detection system, a lane departure detection system, a speed sensor, or a weather sensor.

In Example 72, the subject matter of any one or more of Examples 66-71 optionally include wherein adaptively adjusting the notification comprises: means for preparing the notification in a first state for presentation to the vehicle operator, the first state including a first mode; and means for presenting the notification in a second state to the vehicle operator based on a change in the operating context, the second state including a second mode.

In Example 73, the subject matter of Example 72 optionally includes wherein the first mode and second mode includes haptic, visual, or audio output.

In Example 74, the subject matter of any one or more of Examples 72-73 optionally include wherein the second mode includes an adjustment to a notification intensity level of the first mode.

In Example 75, the subject matter of any one or more of Examples 72-74 optionally include wherein the second mode includes disablement of the first mode.

In Example 76, the subject matter of any one or more of Examples 72-75 optionally include wherein the first mode and the second mode includes output to a heads up display, a steering wheel of the vehicle, a seat of the vehicle, a lane departure warning system of the vehicle, a collision detection system of the vehicle, an audio device of the vehicle, or a display of the vehicle.

In Example 77, the subject matter of any one or more of Examples 72-76 optionally include wherein presenting the notification in the second state to the vehicle operator based on a change in the operating context comprises: means for determining a direction of gaze of the vehicle operator; and means for presenting the notification in the second state to the vehicle operator based on the direction of gaze.

In Example 78, the subject matter of any one or more of Examples 72-77 optionally include wherein presenting the notification in the second state to the vehicle operator based on a change in the operating context comprises: means for determining a load on the vehicle; and means for presenting the notification in the second state to the vehicle operator based on the load on the vehicle.

In Example 79, the subject matter of any one or more of Examples 66-78 optionally include wherein adaptively adjusting the notification comprises: means for preparing the notification in a first state for presentation to the vehicle operator, the first state including a first mode; and means for presenting the notification in a second state to the vehicle operator based on the identity of the vehicle operator, the second state including a second mode.

In Example 80, the subject matter of Example 79 optionally includes wherein presenting the notification in the second state to the vehicle operator based on the identity of the vehicle operator comprises: means for determining a familiarity of the vehicle operator with a location of the vehicle using the identity of the vehicle operator; and means for presenting the notification in the second state to the vehicle operator based on the familiarity of the vehicle operator with the location of the vehicle.

In Example 81, the subject matter of any one or more of Examples 79-80 optionally include wherein presenting the notification in the second state to the vehicle operator based on the identity of the vehicle operator comprises: means for determining a vehicle operation experience level of the vehicle operator using the identity of the vehicle operator; and means for presenting the notification in the second state to the vehicle operator based on the vehicle operation experience level of the vehicle operator.

In Example 82, the subject matter of any one or more of Examples 79-81 optionally include wherein the first mode and second mode includes haptic, visual, or audio output.

In Example 83, the subject matter of any one or more of Examples 79-82 optionally include wherein the second mode includes an adjustment to a notification intensity level of the first mode.

In Example 84, the subject matter of any one or more of Examples 79-83 optionally include wherein the second mode includes disablement of the first mode.

In Example 85, the subject matter of any one or more of Examples 66-84 optionally include wherein the operating context includes a set of environmental attributes that define an environment in which the vehicle is operating and a set of operator attributes that define a current state of the vehicle operator.

In Example 86, the subject matter of any one or more of Examples 80-85 optionally include wherein the means for determining the familiarity of the vehicle operator comprises means for analyzing operator profile data from a profile database to determine how long the vehicle operator has been operating vehicles.

The above detailed description includes references to the accompanying drawings, which form a part of the detailed description. The drawings show, by way of illustration, specific embodiments that may be practiced. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

All publications, patents, and patent documents referred to in this document are incorporated by reference herein in their entirety, as though individually incorporated by reference. In the event of inconsistent usages between this document and those documents so incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first.” “second,” and “third.” etc. are used merely as labels, and are not intended to impose numerical requirements on their objects.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is to allow the reader to quickly ascertain the nature of the technical disclosure and is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the embodiments should be determined with reference to the appended claims, along with the full scope of equivalents to which such claims are entitled.

What is claimed is:

1. A system for context derived driver assistance, the system comprising:
at least one processor; and
a memory including instructions that, when executed by the at least one processor, cause the at least one processor to perform operations to:
determine an identity of a vehicle operator of a vehicle;
determine an operating context of the vehicle; and
adaptively adjust a notification concerning the operating context based on the identity of the vehicle operator.

2. The system of claim 1, the operations to determine the identity of the vehicle operator comprising operations to:
receive a signal from a key fob used in operating the vehicle; and
determine the identity of the vehicle operator based on an association between the key fob and the vehicle operator.

3. The system of claim 1, the operations to determine the identity of the vehicle operator comprising operations to:
capture an image of an operating position of the vehicle;
evaluate the image to identify an object in the image; and
determine the identity of the vehicle operator based on the object in the image matching a face of the vehicle operator.

4. The system of claim 1, the operations to determine the identity of the vehicle operator comprising operations to:
receive biometric data from a biometric sensor of the vehicle; and
determine the identity of the vehicle operator based on a match between the received biometric data and biometric data of the vehicle operator.

5. The system of claim 1, the operations to determine the operating context of the vehicle comprising operations to:
receive a set of sensor data from the vehicle; and
determine the operating context of the vehicle using the set of sensor data.

6. The system of claim 1, the operations to adaptively adjust the notification comprising operations to:
prepare the notification in a first state for presentation to the vehicle operator, the first state including a first mode; and
present the notification in a second state to the vehicle operator based on a change in the operating context, the second state including a second mode.

7. The system of claim 6, the operations to present the notification in the second state to the vehicle operator based on a change in the operating context comprising operations to:
determine a direction of gaze of the vehicle operator; and
present the notification in the second state to the vehicle operator based on the direction of gaze.

8. The system of claim 6, the operations to present the notification in the second state to the vehicle operator based on a change in the operating context comprising operations to:
determine a load on the vehicle; and
present the notification in the second state to the vehicle operator based on the load on the vehicle.

9. The system of claim 1, the operations to adaptively adjust the notification comprising operations to:
prepare the notification in a first state for presentation to the vehicle operator, the first state including a first mode; and
present the notification in a second state to the vehicle operator based on the identity of the vehicle operator, the second state including a second mode.

10. The system of claim 9, the operations to present the notification in the second state to the vehicle operator based on the identity of the vehicle operator comprising operations to:
determine a familiarity of the vehicle operator with a location of the vehicle using the identity of the vehicle operator; and
present the notification in the second state to the vehicle operator based on the familiarity of the vehicle operator with the location of the vehicle.

11. At least one machine readable medium including instructions for context derived driver assistance that, when executed by a machine, cause the machine to perform operations to:
determine an identity of a vehicle operator of a vehicle;
determine an operating context of the vehicle; and
adaptively adjust a notification concerning the operating context based on the identity of the vehicle operator.

12. The at least one machine readable medium of claim 11, the operations to determine the operating context of the vehicle comprising operations to:
receive a set of sensor data from the vehicle; and
determine the operating context of the vehicle using the set of sensor data.

13. The at least one machine readable medium of claim 12, wherein the set of sensor data includes data from a global positioning sensor, an accelerometer, a gyroscope, a magnetometer, a collision detection system, a lane departure detection system a speed sensor, or a weather sensor.

14. The at least one machine readable medium of claim 11, the operations to adaptively adjust the notification comprising operations to:
prepare the notification in a first state for presentation to the vehicle operator, the first state including a first mode; and
present the notification in a second state to the vehicle operator based on a change in the operating context, the second state including a second mode.

15. The at least one machine readable medium of claim 11, the operations to adaptively adjust the notification comprising operations to:
prepare the notification in a first state for presentation to the vehicle operator, the first state including a first mode; and
present the notification in a second state to the vehicle operator based on the identity of the vehicle operator, the second state including a second mode.

16. A method for context derived driver assistance, the method comprising:
determining an identity of a vehicle operator of a vehicle using profile data from a profile database;
determining an operating context of the vehicle using environmental data, the environmental data including sensor data; and
adaptively adjusting an electronic notification concerning the operating context based on the identity of the vehicle operator.

17. The method of claim 16, wherein determining the identity of the vehicle operator comprises:
receiving a signal from a key fob used in operating the vehicle; and
determining the identity of the vehicle operator based on an association between the key fob and the vehicle operator.

18. The method of claim 16, wherein adaptively adjusting the electronic notification comprises:
preparing the electronic notification in a first state for presentation to the vehicle operator, the first state including a first mode; and
presenting the electronic notification in a second state to the vehicle operator based on a change in the operating context, the second state including a second mode.

19. The method of claim 18, wherein the second mode includes an adjustment to a notification intensity level of the first mode.

20. The method of claim 18, wherein the second mode includes disablement of the first mode.
